(19) United States
(12) Patent Application Publication
(10) Pub. No.: US 2004/0197221 A1
(43) Pub. Date: Oct. 7, 2004

(54) Artificial christmas tree

(76) Inventor: Virgil E. Stanley III, Indianapolis, IN (US)

Correspondence Address: COATS & BENNETT, PLLC, P O BOX 5, RALEIGH, NC, 27602, US

(21) Appl. No.: 10/797,490

(22) Filed: Mar. 10, 2004

Related U.S. Application Data
(63) Continuation-in-part of application No. 10/756,224, filed on Jan. 13, 2004.
Application No. 10/756,224 is a continuation-in-part of application No. 10/164,818, filed on Jun. 7, 2002.

(51) Int. Cl. A61L 9/04; A61L 9/02

(52) U.S. Cl. 422/5; 428/19; 428/18; 422/124; 422/125

(57) ABSTRACT

An artificial Christmas tree includes a trunk and a series of branches extending outwardly from the trunk. A fragrance source is associated with the artificial Christmas tree. In one embodiment, the fragrance source is disposed within a container contained within the Christmas tree or disposed adjacent the Christmas tree. Air is directed past the fragrance source where the fragrance is mixed with the air to form an air-fragrance mixture. This air-fragrance mixture is directed upwardly through the trunk of the artificial Christmas tree and eventually emitted from the Christmas tree. Preferably the fragrance would be selected such that the scent emitted would simulate a live Christmas tree.

### CROSS REFERENCE TO RELATED APPLICATIONS

This is a continuation-in-part of U.S. patent application Ser. No. 10/756,224 filed Jan. 13, 2004, which was in turn a continuation-in-part of U.S. patent application Ser. No. 10/164,818 filed on Jun. 7, 2002.

### FIELD OF THE INVENTION

The present invention relates to artificial Christmas tree and more particularly to an artificial tree having a fragrance source associated therewith.

### BACKGROUND OF THE INVENTION

Artificial Christmas trees are well known and are sold and used throughout the world. What is truly significant about artificial Christmas trees today is how realistic and beautiful artificial Christmas trees can be and still be marketed at affordable prices. However, one of the main drawbacks to artificial Christmas trees is that they do not have the pleasing scent and aroma that is ordinarily associated with real Christmas trees. Therefore, there has been and continues to be a need for an artificial Christmas tree that is provided with some means that produces or generates a scent or fragrance that simulates living Christmas trees.

### SUMMARY OF THE INVENTION

An exemplary embodiment of the present invention entails an artificial Christmas tree comprising a Christmas tree having a hollow trunk or stem. A fragrance source is provided and emits a fragrance that is directed from the artificial Christmas tree.

Further, the present invention entails a method of generating or directing a fragrance that is associated with an artificial Christmas tree having a hollow stem and a Christmas tree portion. The method entails locating a fragrance source in a Christmas tree portion detachably coupled to a hollow stem and emitting a fragrance from the fragrance source.

Further, the present invention entails a method of generating or directing a fragrance that is associated with artificial potpourri. The method entails aggregating a plurality of artificial Christmas tree portions and a fragrance source in one or more of the plurality of Christmas tree portions to emit a fragrance from the artificial potpourri.

Other objects and advantages of the present invention will become apparent and obvious from a study of the following description and the accompanying drawings, which are merely illustrative of such invention.

### BRIEF DESCRIPTION OF THE DRAWINGS

FIG. 1 is a sectional view of the artificial flower of the present invention showing one embodiment of the present invention where a fragrance source is disposed in the hollow stem of the flower.

FIG. 1A is an enlarged fragmentary sectional view of a portion of the stem of the artificial flower having the fragrance source therein.

FIG. 2 is a sectional view similar to FIG. 1 but with a different fragrance source than that shown in FIG. 1.

FIG. 2A is an enlarged sectional view of a potion of the stem of the artificial flower shown in FIG. 1 having the fragrance source disposed therein.

FIG. 3 is a view similar to FIGS. 1 and 2, but illustrating another fragrance source for the artificial flower.

FIG. 3A is an enlarged sectional view of the portion of the stem of the artificial flower shown in FIG. 3 and which shows the fragrance source disposed therein.

FIG. 4 illustrates an artificial flower similar to the one shown in FIG. 1, but with a power supply externally connected to a bulb portion of the stem.

FIG. 4A is an enlarged sectional view of the stem of the artificial flower shown in FIG. 4 that shows the fragrance source disposed therein.

FIG. 5 is a sectional view of an alternate design for an artificial flower where a fragrance source is disposed in a detachably coupled flower portion.

FIG. 5A is a sectional view of the flower portion of FIG. 5.

FIG. 6 illustrates a partial cross-section of an artificial flower arrangement.

FIG. 7 illustrates artificial potpourri according to an exemplary embodiment of the present invention.

FIG. 8 illustrates a sectional view of another exemplary embodiment of artificial potpourri according to the present invention.

FIG. 8A is an enlarged sectional view of a potpourri flower of FIG. 8.

FIG. 9 illustrates artificial potpourri according to another exemplary embodiment of the present invention.

FIG. 9A illustrates an enlarged sectional view of a potpourri flower of FIG. 9.

FIG. 10A illustrates an artificial flower disposed within a car according to an exemplary embodiment of the present invention.

FIG. 10B illustrates another artificial flower disposed within a car according to an exemplary embodiment of the present invention.

FIG. 10C illustrates another artificial flower disposed within a car according to an exemplary embodiment of the present invention.

FIG. 11 is a front elevational view of an artificial Christmas tree having the capability of emitting a fragrance or scent that simulates that emitted by a live Christmas tree.

FIG. 12 is a fragmentary perspective view showing one embodiment that illustrates branches that are connectable to the trunk of the Christmas tree and further illustrates a Christmas tree supported within a container equipped with a fragrance source and a fan for directing air past the fragrance source and into the Christmas tree.

### DESCRIPTION OF EXEMPLARY EMBODIMENTS OF THE INVENTION

With further reference to the drawings, the artificial flower of the present invention is shown therein and indicated generally by the numeral 10. The artificial flower 10 includes a hollow stem indicated generally by the numeral 12 and a flower portion 16 secured to or extending from the upper portion of the stem 12. In the context of this application, the term “artificial” simply means non-living. Thus, the artificial flower 10 can be made of various materials such as plastics, metal, synthetic materials, or could comprise dried flowers or dried vegetation.

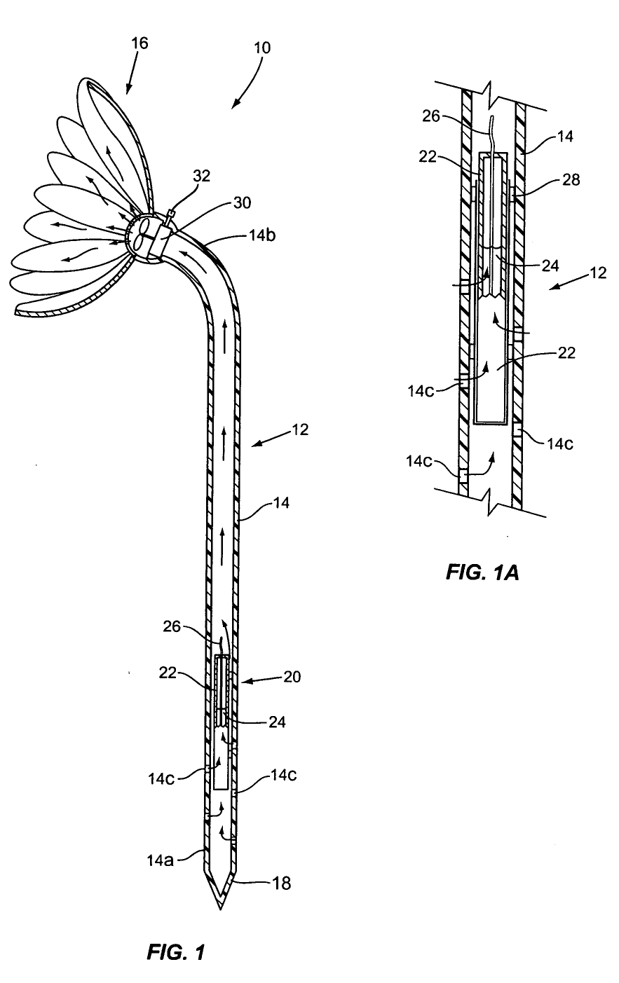

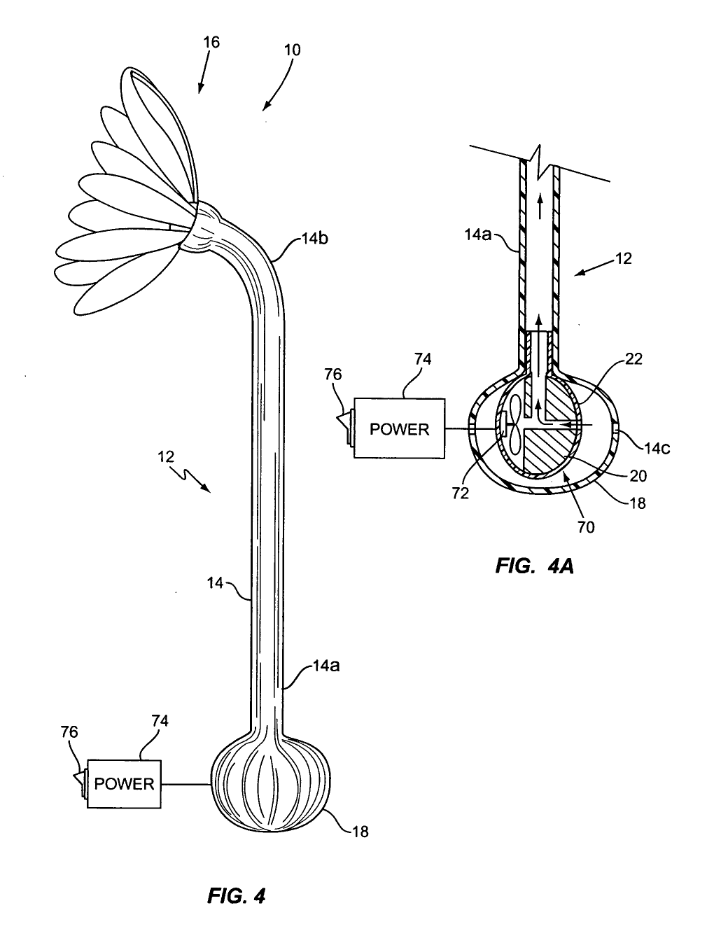

As seen in the drawings, the stem 12 is hollow. Stem 12 includes a surrounding wall structure 14 and a lower portion 14a that extends to an anchor end 18. As seen in FIGS. 1-3, anchor end 18 is formed into a point that permits the artificial flower 10 to be staked or spiked into a support material such as Styrofoam, potting soil, dirt or other supporting structure. Alternately, anchor end 18 may be formed into a bulb shape (see FIG. 4) similar to that of a real flower.

Opposite the lower portion 14a is an upper portion 14b. It is noted that the upper portion 14b of stem 12 is disposed adjacent the flower portion 16. The end of stem 12 about the upper portion 14b may be open or partially closed. In the embodiment illustrated in FIGS. 1-3, the end of the upper portion 14b of the stem 12 includes a rounded end that includes a series of openings formed therein. As will be described subsequently herein, the stem is designed such that air and a fragrance can move therethrough and, in at least one embodiment, is designed such that the fragrance can be emitted or dispersed from the upper portion 14b of the stem 12 into an environment where the flower portion 16 of the artificial flower 10 resides. Also, it is appreciated that the wall structure 14 of the stem 12 may include one or more openings 14c along the length of stem 12. Again, as will be appreciated from subsequent portions of this disclosure, openings 14c within the stem 12 may permit air to enter the stem 12 and move upwardly through the hollow stem 12 towards the flower portion 16.

Flower portion 16 is disposed adjacent the upper portion 14b of the stem 12. Again, the flower portion 16 forms a part of the artificial flower 10 and in the particular embodiments illustrated herein, the flower portion 16 extends from the upper portion of the stem. It is appreciated that flower portion 16 may be secured or integrally formed with the stem 12 through various manufacturing and fabrication techniques. Flower portion 16 may also assume various shapes and configurations. In some embodiments, it is contemplated that the flower portion 16, as illustrated in the drawings, would form a generally cup shape and comprise a series of petals.

The present invention entails associating a fragrance source, indicated generally by the numeral 20, with the artificial flower 10 for dispersing fragrance into the environment. The fragrance source 20 can be of various conventional types. Further, the particular scent emitted by the fragrance source 20 may vary and may be selected to simulate or mimic the smell or scent of various flowers. In addition, the size of the fragrance source 20 and/or the size of the air intake openings and/or outtake openings may affect the amount of fragrance dispersed into the environment.

Those skilled in the art will appreciate that the consumer may control any or all of these parameters and characteristics. For example, a consumer may select a particular scent by selecting one or more fragrance sources 20 for one or more artificial flowers 10. The consumer may also control the amount of dispersed fragrance by opening a slidable panel (not shown) to expose more openings in the container 22 or by varying the size of the intake openings. In addition, the amount or size of the fragrance source 20 can be varied. For example, in embodiments utilizing a solid fragrance source, the present invention may employ various sizes of such fragrance sources 20. In cases where a liquid or a semi-liquid fragrance is used, the quantity of the fragrance source 20 held within the artificial flower 10 can be varied.

In the embodiment illustrated in FIG. 1, fragrance source 20 comprises a container 22 having a selected liquid fragrance 24 contained therein. Container 22 is preferably sealed but includes an opening for receiving a wick 26. Wick 26 extends downwardly into container 22 and includes a portion that is submerged within the liquid fragrance 24. A portion of the wick 26 extends from the top of the container 22 and is exposed. Container 22 can be disposed in various locations about the artificial flower 10. In the embodiment illustrated in FIG. 1, container 22 is disposed within the hollow stem 12.

Various mounting structures or mounting techniques can be utilized. For example, the container 22 can be set or held in an open mounting structure 28 that is frictionally supported between the interior walls of the stem 12. In the case of the embodiment shown in FIG. 1, the open mounting structure 28 is of an open plastic frame that basically slides into the stem 12 and is frictionally held therein. It will be appreciated that the open mounting structure 28 may alternatively be secured to the walls 14 of the stem 12 by adhesive or by any other known securing means. It may also be beneficial in certain embodiments for the mounting structure 28 to be designed such that there is formed at least one air passageway between the container 22 and the interior walls of the stem 12. In other words, it will be beneficial in certain embodiments to provide an open space between the container 22 and the interior walls to allow air to pass upwardly around the container 22 and over the wick 26.

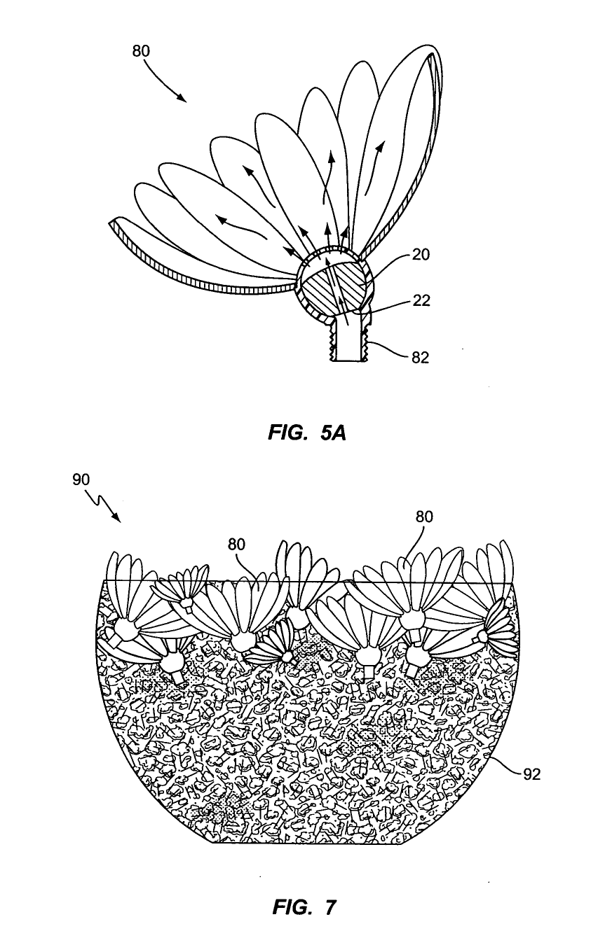

In the case of the embodiment shown in FIG. 1, there is provided a fan 30 or impeller disposed in the upper end portion 14b of the stem 12. The fan 30 is a battery-powered fan that includes a main body held and supported within the upper portion 14b of the stem and including a fan blade, impeller, or propeller extending therefrom. A switch 32 may extend from the main body of the fan 30 outwardly through a sidewall of the stem 12. While not shown, those skilled in the art will appreciate that the power source 74 for the fan 30 may be disposed internally or externally to the artificial flower 10. Further, as shown in FIG. 6 and discussed further below, anchor end 18 may electrically connect to an external power source when staked or held within support material. In addition, alternate embodiments of the present invention may also include power adapters so that fan 30 may plug into a wall outlet (not shown) or a vehicle power port.

According to the present invention, fan 30 may provide a fixed airflow rate. Alternatively, fan 30 may provide a variable airflow rate. For example, fan 30 may be a multi-speed fan that enables a consumer to select the amount of fragrance dispersed into the environment by selecting a fan speed. In a preferred embodiment, a consumer selects a fan speed by positioning switch 32 in the desired position.

In the case of the design shown in FIG. 1, the fan 30 is disposed above the fragrance source 20. Therefore, the impeller or fan blade associated with the fan 30 is designed to induce or pull air from below the container 22, past the container 22 and over the wick of 26. The fan causes air to be induced through the openings 14c into the interior of the stem 12. Once in the stem 12, the induced air is pulled upwardly past the container 22 and the wick 26. Accordingly, fragrance on the saturated or wet wick 26 will be transferred to the passing air and ultimately will be dispersed out the upper portion 14b of the stem 12 adjacent the flower portion 16. Note also that the main body or frame of the fan 30 would be provided with openings that would enable air to be moved or pulled through the upper portion 14b of the stem 12, through the fan structure, and out of the upper end of the stem 12 to where the fragrance-laden air disperses into an area occupied in part at least by the flower portion 16.

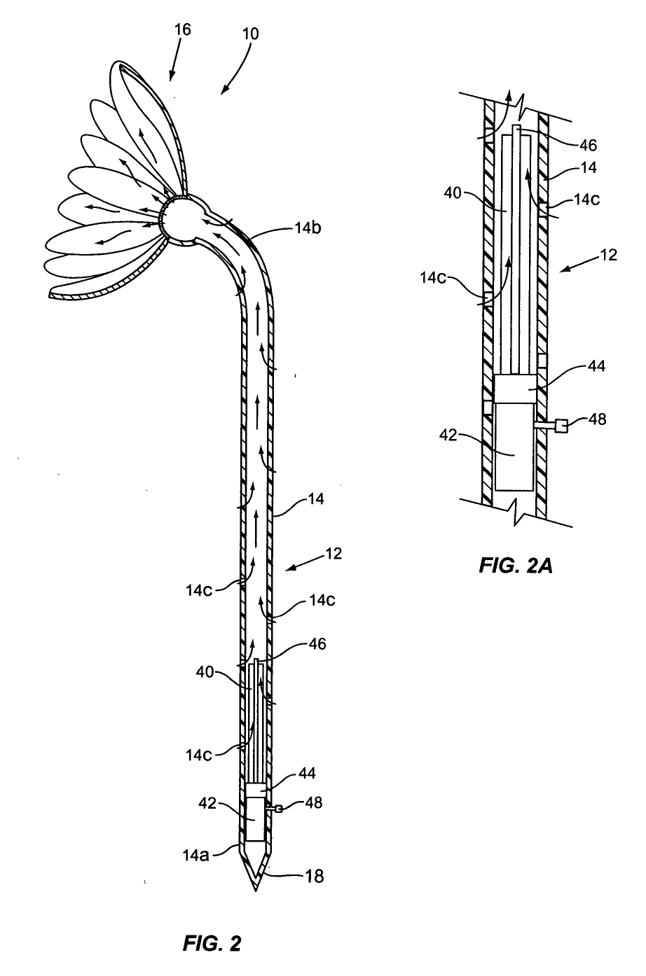

Turning to FIG. 2, an alternative embodiment for the artificial flower 10 is shown therein. In this case, the fragrance source 20 is in the form of a fragrance gel or block 40. It will be appreciated that fragrance blocks are known in the air freshener art and therefore, details of such will not be submitted herein because those skilled in the art will understand the basic structure and composition of conventional fragrance blocks. For example, see U.S. Pat. No. 6,289,176, the disclosure of which is expressly incorporated herein by reference. In any event, fragrance block 40 is disposed within the stem 12 of the artificial flower, as shown in the embodiment of FIG. 2. The fragrance block 40 may assume different configurations. In the case of the embodiment illustrated herein, fragrance block 40 is elongated and round and is in the form of a generally cylindrical shape. Further, fragrance block 40 includes a central opening.

The artificial flower 10 may also include a heater 42, where fragrance block 40 is supported in the stem 12 over the heater 42. Heater 42 may be battery-powered and may include a switch 48 that extends from the heater 42 out the sidewall of the stem 12. As with the fans described above, heater 42 includes a power source (not shown) that may reside within or externally from the artificial flower 10 and may include a power adapter (not shown) to allow heater 42 to plug into a wall outlet. Further, as with the fans 30 described above, heater 42 may provide a fixed amount of heat or may provide a variable amount of heat based on the position of switch 48. A mounting block 44 disposed over the heater and a heating element 46, such as resistive heating element, extends upwardly from the heater 42 and the mounting block 44 and extends through the central opening formed in the fragrance block 40.

Further, the side wall structure 14 of the stem 12, especially in the area adjacent the position of the fragrance block 40, will includes a series of openings 14c therein to allow air to be induced or to naturally flow into the stem 12. That is, the fragrance block 40 would be preferably spaced inwardly from the wall structure 14 of the stem 12 so as to allow air to pass between the fragrance block 40 and the interior walls of the stem 12. Additionally, a fan 30, such as shown in FIG. 1, can be positioned below or above the fragrance block 40 to induce air into the stem 12 and over the fragrance block 40. When the heater 42 is turned on, heating element 46 heats the fragrance block 40 and cause the fragrance block 40 to vaporize and produce a vaporized scent or aroma.

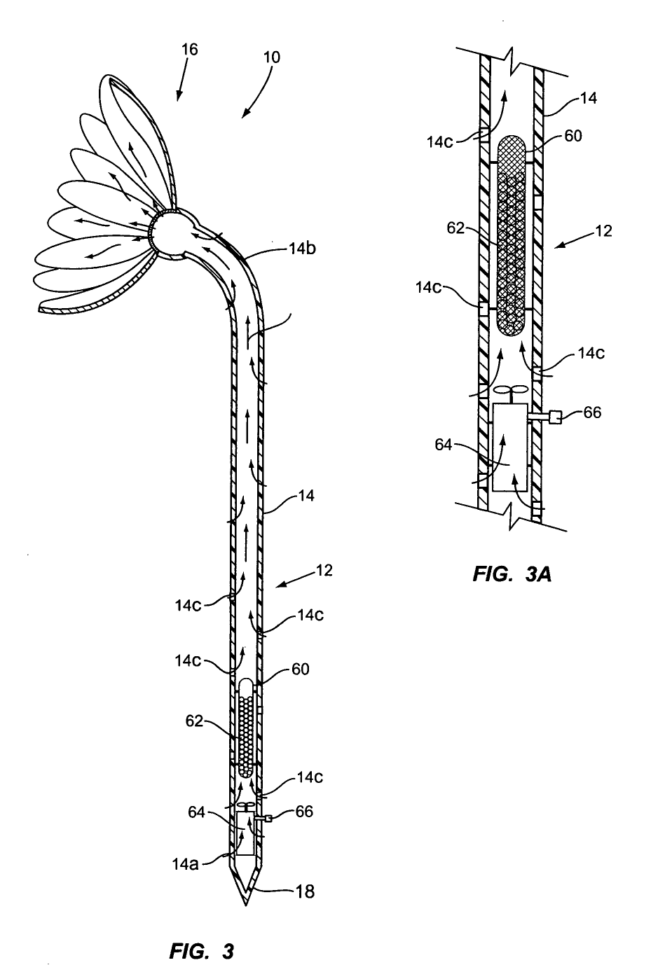

Turning now to FIG. 3, another embodiment of the present invention is shown therein. In this case, container 22 is a permeable container 60. Permeable container 60 may assume various forms. For example, permeable container 60 may comprise a plastic container with openings formed therein that enable air to circulate through the permeable container 60. Disposed within the permeable container 60 is a fragrance source 20, such as a fragrance gel or an array of fragrance pellets 62. Each fragrance pellet 62 may comprise a fragrance particle or ball and would over time emit a desired fragrance. Preferably the permeable container 60, including the fragrance pellets 62, would be supported within a mounting structure secured in the stem 12. In one embodiment, the mounting structure is similar to that discussed above with respect to the mounting structure utilized to hold and support the container 22. In any event, the function of the mounting structure would be able to hold the permeable container 60 within the stem 12. Preferably the mounting structure would be of an open frame design that would, when inserted within the stem 12, provide an opening around the permeable container 60 to allow air to flow past. In some embodiments, it is foreseen that the permeable container 60 would be frictionally retained or held within the stem 12 and would effectively assume substantially the entire cross section of the stem about a selected length of the stem. In this case, air moving from below the permeable container 60 upwardly would be forced to pass through the permeable container 60.

In any event, the design of FIG. 3 may also include a fan 64 with a propeller, impeller, or blade associated therewith. Fan 64 in this embodiment is disposed below the permeable container 60, however, it is understood that the fan 64 could be oriented above the permeable container 60, for example, as indicated in FIG. 1. Fan 64 may include a switch 66 that extends outwardly therefrom through the wall structure 14 of the stem 12. Again, as was the case with the fan shown in FIG. 1, fan 64 may be battery-powered and the main body of the fan 64 would generally be of an open construction that allows air to be pulled from below the fan 64, through the open main body of the fan 64, and upwardly through the stem 12.

Still another embodiment of an artificial flower of the present invention is illustrated in FIG. 4. As with the embodiments described above, artificial flower 10 includes the hollow stem 12 and the flower portion 16 secured to or extending from the upper end of the hollow stem 12. In this embodiment, however, anchor end 18 of stem 12 forms the shape of a bulb. Therefore, it can be said that the artificial flower 10 includes a lower bulb portion 18.

Bulb portion 18 secures to the stem 12 at the base of the lower portion 14a of stem 12 by any means known in the art, including friction and/or by the use of adhesives and/or fastening devices. Further it is contemplated that the bulb portion 18 could be integrally formed with the stem 14. The bulb portion 18 forms an internal cavity, indicated generally by the numeral 70. Disposed within the cavity 70 is a housing structure 22 that includes a diffuser or fan 72 and a fragrance source 20. While the illustrated embodiment illustrates a fan 72, those skilled in the art will appreciate that a heater may be used in place of or in addition to the fan 72.

Formed in the housing 22 is an air passage that, as seen in FIG. 4A, allows air to enter the housing 22 and pass through and into contact with the fragrant source 20 after which the air is directed through the hollow stem 14. It is noted in FIG. 4A that the bulb portion 18 includes at least one air inlet 14C for permitting air to enter the internal cavity 70. Fan 72 electrically connects with a power source 74 that includes an on/off switch 76. In a preferred embodiment, the power source 74 is a battery power source. However, power source 74 may include a power adaptor that can be plugged into an electrical outlet.

When switched on, the power source activates the fan 72 such that air circulates through and around fragrance source 20. As described above, fan 72 may provide a fixed rate airflow or a variable rate airflow. The scented air then travels through the hollow stem 12 and exits the artificial flower 10 at the flower portion 16. While the embodiment illustrated in FIGS. 4 and 4A shows a container 22 that includes both the fan 72 and the fragrance 20, those skilled in the art will appreciate that the present invention is not so limited. For example, fan 72 may be housed separately from the container 22 within anchor end 18.

In the embodiment illustrated in FIGS. 4 and 4A, fragrance source 20 comprises a fragrance block or gel. However, the present invention is not so limited. Container 22 may contain any known fragrance source 20, including the fragrance sources described above. For example, container 22 may contain a liquid fragrance 24, where a wick 26 positioned in the liquid fragrance 24 extends from the fragrance container 72 (similar to FIG. 1). Alternately, container 22 may comprise a permeable container 60 with fragrance gel or pellets 62 disposed therein (similar to FIG. 3).

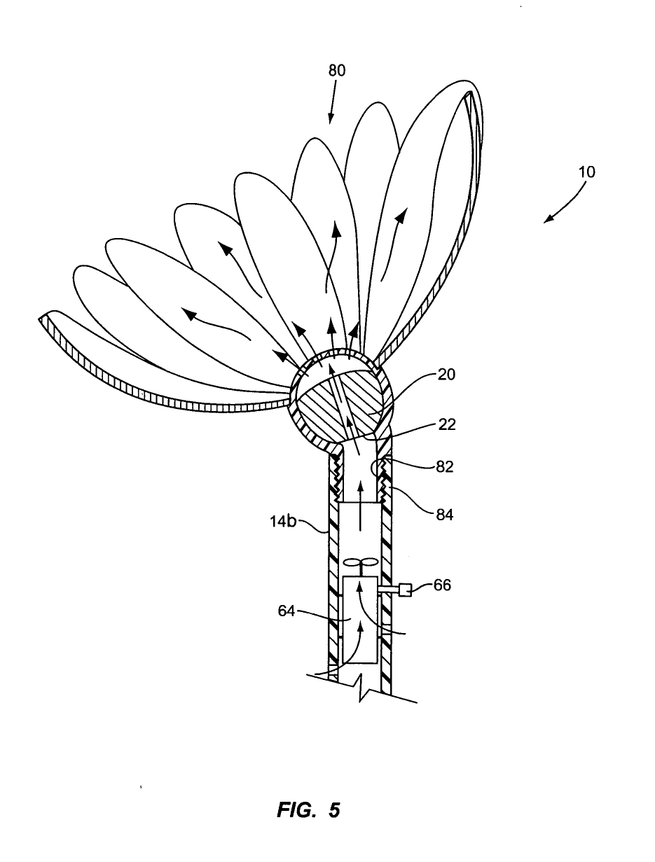

Referring now to FIG. 5, another embodiment of the invention will be described herein. In the illustrated embodiment, artificial flower 10 comprises a flower portion 80 that detachably couples to the hollow stem 12. Flower portion 80 also includes a fragrance source 20 that may be disposed in a container 22 secured within flower portion 80 by any of the means described above. As a result, the fragrance provided by fragrance source 20 may be dispersed to the surrounding environment through evaporation and natural airflow.

Alternatively, a diffusion source, such as a fan 64 and/or a heater (not shown), may be positioned within the artificial flower to disperse the scented air. For example, fan 64 may be disposed within the upper end 14b of the hollow stem 12 proximate the flower portion 80. As described above, the fan induces airflow around and/or through the fragrance source, causing scented air to be emitted from the flower portion 80.

As seen in FIG. 5A, the detachable flower portion 80 includes a base that is generally disposed about the bottom of the flower portion 80. As will be discussed below, the base portion lies just above a fastener or connector that is adapted to attach the flower portion 80 to the stem 12 of the artificial flower 10. In any event, as seen in FIG. 5A, the base portion of detachable flower portion 80 includes a hollow cavity for receiving and holding a container or housing 22 that contains or holds the fragrance source 20. Note in FIG. 5A where at least one air passageway extends through the container 22 and through the fragrance source 20. Note also that the upper portion of the base, above the fragrance source 20, includes a series of openings for dispersing the air among the petals of the detachable flower portion 80.

As mentioned above, flower portion 80 detachably couples to stem 12. Typically, a connector 82 disposed on a bottom end of the flower portion 80 detachably couples to a corresponding connector 84 disposed on the upper end 14b of stem 12. In an exemplary embodiment, connector 82 comprises a threaded section at the base of flower portion 80 that threadably connects to a corresponding threaded section of a connector 84 at the top of the upper end 14b of stem 12. Of course, alternate connectors, such as snaps, straps, etc., may be used. Further, flower portion 80 may simply secure to stem 12 via friction.

Because flower portion 80 detachably couples to the stem 12, a consumer may replace or change the fragrance source 20 at any time simply by removing the former flower portion 80 and attaching a new flower portion 80, which includes a new fragrance source 20, to the stem 12. Alternatively, the consumer may refill container 22 with a new fragrance source 20. In still another embodiment, the consumer may remove the container 22 from the flower portion 80 and couple a new container 22 within flower portion 80. In any event, the artificial flower 10 of the present invention allows the consumer to exchange and/or replenish the fragrance source 20 of artificial flower 80 without replacing the entire flower 10.

The artificial flowers 10 of the present invention may be used individually or may be arranged as an artificial flower bouquet 90 within a container 92, such as a vase or bowl, as shown in FIG. 6. FIG. 6 shows a plurality of artificial flowers 10 secured within a support structure 88 disposed within the container 92. The support structure 88 includes one or more air intakes 14c, a diffuser, such as a fan 64, and a power source 74 that electrically connects to the diffuser and optionally includes a switch 76. In one embodiment, each artificial flower 10 includes a fragrance source 20, as shown in any one of FIGS. 1-3. The artificial flowers then emit a fragrance when the fan 64 pushes air through the hollow stems 14 and across the fragrance sources 20 disposed within the artificial flowers.

In an alternate embodiment, the fragrance source 20 may be disposed within support structure 88 proximate the air intake 14c. In this embodiment, the air is scented by fragrance source 20 before entering the fan 64. Fan 64 provides enough airflow to push the scented air through the hollow stems 14 of the artificial flowers 10 such that scented air is emitted from the bouquet of artificial flowers 10.

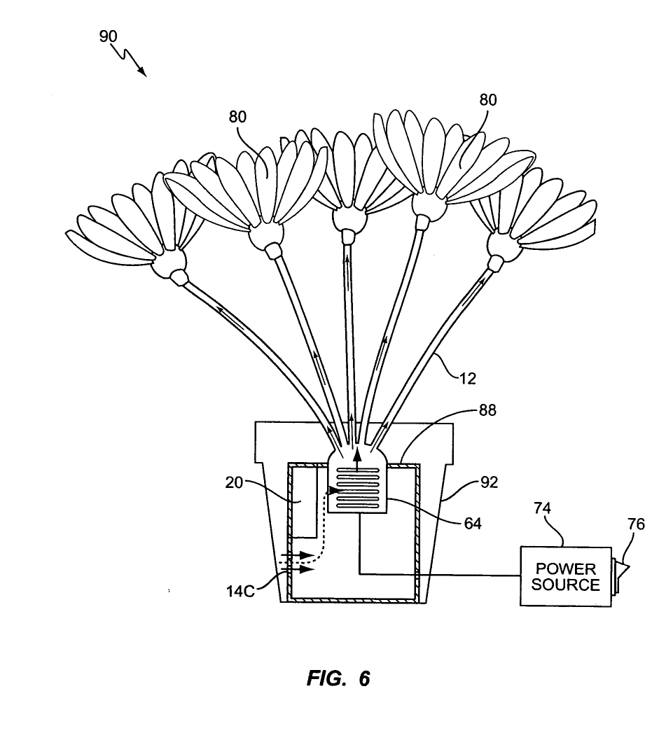

FIG. 7 illustrates yet another embodiment of the present invention where a collection of the flower portions 80 shown in FIG. 5A may be used as potpourri 90. While FIG. 5A shows a flower portion 80 that includes the connector 82, it will be appreciated that some embodiments of the present invention may exclude the connector 82 when the flower portions 80 are used for potpourri.

In any event, potpourri 90 may be placed in any suitable container 92, as shown in FIG. 7, or may be scattered loosely on any surface. Potpourri 90 may also include additional artificial flowers or material that operates as filler and does not contribute to the fragrance being emitted from the potpourri 90. When collected as potpourri, each flower portion 80 contributes to the overall scent emitting from the potpourri 90. A consumer may therefore create any desired fragrance by either using flower portions 80 with the same desired fragrance or by combining two or more different flower portions 80 with different fragrances.

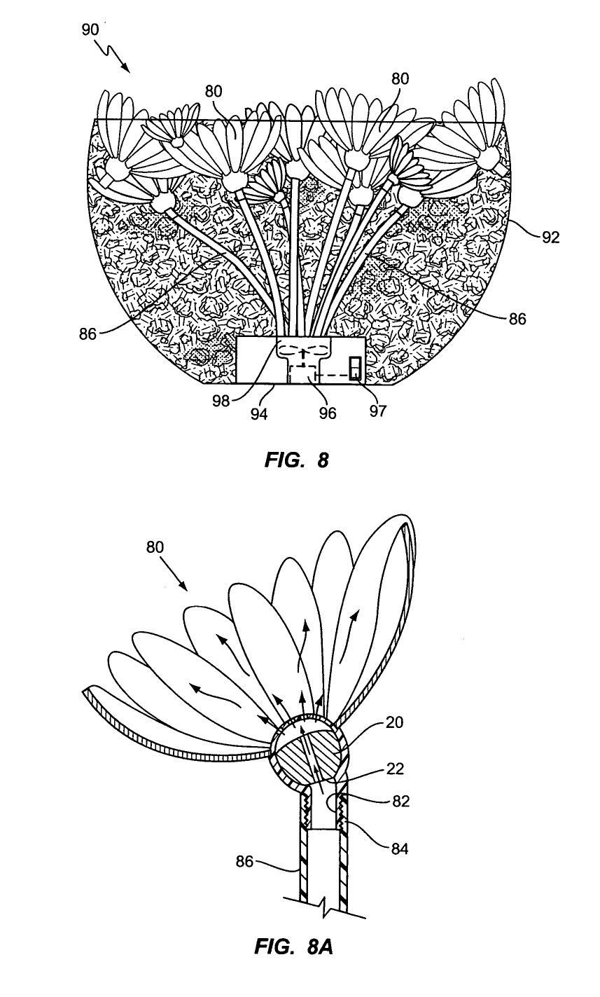

In exemplary embodiments, a scent diffuser 94 may be disposed in a container 92 as shown in FIG. 8. Scent diffuser 94 includes a fan 96 (or any other known diffuser as discussed above) electrically connected to a switch 97. Hollow tubes 86, made of a flexible or rigid material, couple to one or more openings 98 in diffuser 94 at a first end and couple to one or more flower portions 80 at an opposite end. Hollow tubes 86 may be detachably or fixedly coupled to the openings 98 according to any known method. Further, as shown in FIG. 8A, one or more flower portions 80 may detachably couple to the hollow tubes 86. As a result, airflow generated by fan 96 exits the openings 98 in diffuser 94, flows through the hollow tubes 86, circulates around and/or through the fragrance sources 20 disposed in the flower portions 80, and causes scented air to disperse from the potpourri 90.

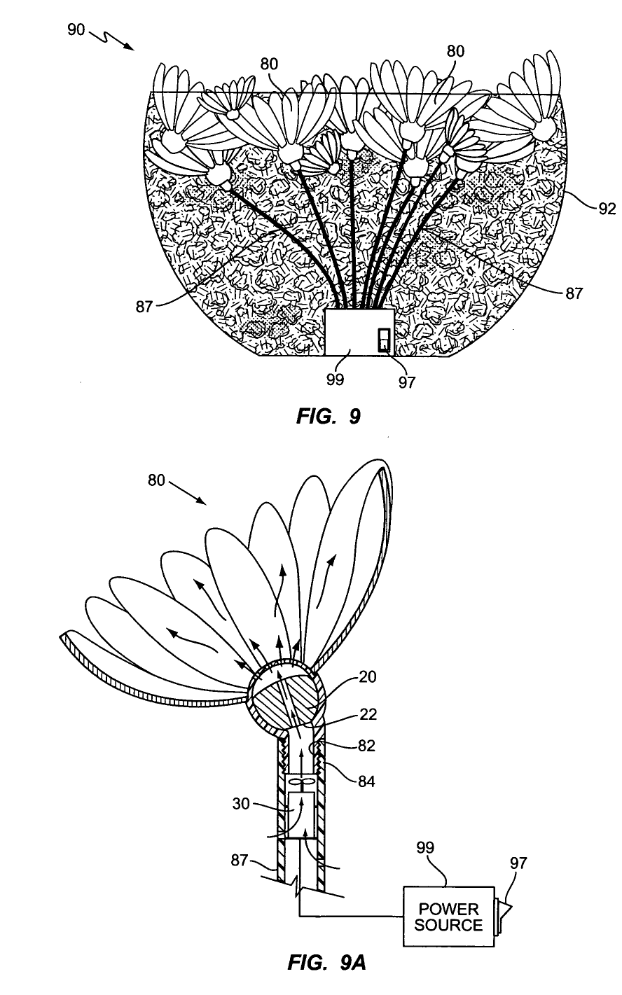

Alternatively, as shown in FIGS. 9 and 9A, each flower portion 80 may include the fragrance and a diffuser, such as a fan 30. In this embodiment, electrical wires 87 detachably connect the flower portions 80 to a power source 99 disposed within the container 92. As discussed above, one or more batteries or a power adapter may make up the power source 99. When the power source 99 is activated, the electrical wires 87 carry the necessary electrical current to power the fans 30 disposed within the flower portions 80, causing air to flow over the fragrance sources 20 and scented air to disperse from the potpourri 90.

Those skilled in the art will appreciate that the flower portions 80 in the potpourri that detachably couple to the hollow tubes 86 or to the electrical wires 87 may be replaced by removing the former flower portion 80 and attaching a new flower portion 80. As a result, a consumer may replace former flower portions 80 with new flower portions 80 any time the consumer wishes to refresh the potpourri scent or to replace the potpourri scent with a new scent.

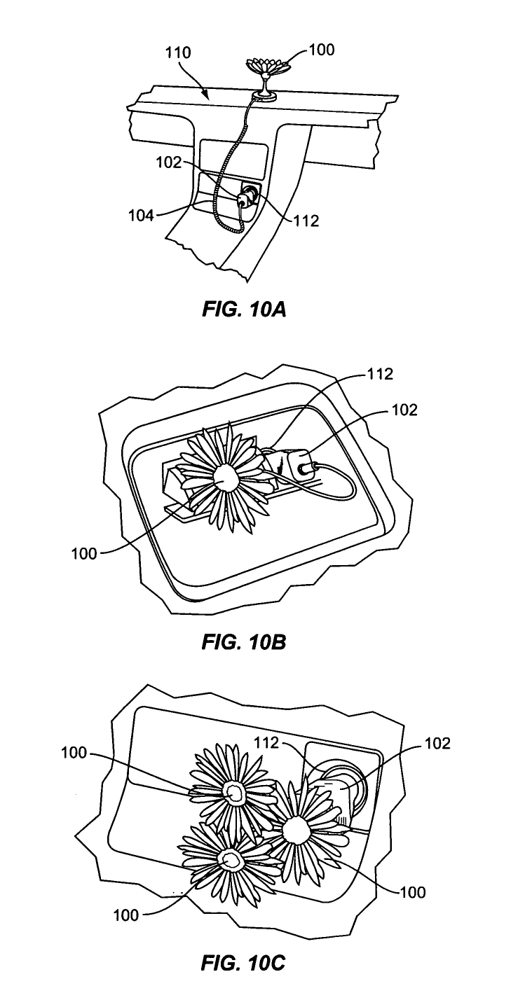

FIGS. 10A-10C illustrate still another embodiment of the present invention. As with the embodiments described above, the artificial flower 100 of FIGS. 10A-10C include a fragrance source and a diffuser, such as a fan or a heater, disposed within the artificial flower 100. However, unlike the embodiments described above, a car battery provides the power for the diffuser via an adapter 102. One end of the adapter 102 detachably couples to the diffuser, while the other end detachably couples to a power socket 112 within a car, such as a cigarette lighter socket, via a power cord 104.

As shown in FIGS. 10A-10B, the artificial flower 100 may include a mounting device or structure for mounting the artificial flower 100 to the dashboard 110 or to an adjacent ashtray. For example, the mount structure may include a suction cup or other semi-permanent mounting device that would function to secure the base of one or more artificial flowers 100 to the dashboard 110, to an ashtray, or to other surfaces found in a vehicle.

FIG. 10C shows another alternate design wherein one or more artificial flowers 100 are integrated together and supported directly or indirectly from an adapter 102 that fits into the cigarette lighter socket 112. Various support structures or devices can be utilized to support the artificial flowers 100 in various configurations.

It is appreciated that the present invention presents an artificial flower that is designed to emit a pleasing fragrance or aroma. A fragrance source associated with the flower is designed to emit a fragrance that will be dispersed about the flower. Although the fragrance source can be disposed in various positions and locations with respect to the artificial flower, in some embodiments, the fragrance source is disposed in the hollow stem that forms a part of the artificial flower. In other embodiments, the fragrance source is disposed in the flower portion of the artificial flower. Moreover, the fragrance source may simply be designed or selected to be of the type that will slowly and over a period of time simply emit a fragrance that will move through or from the artificial flower. Dispersion of the fragrance or scent can be enhanced by utilizing a heater to heat the fragrance source and/or a fan to pull or push air past the fragrance source.

The present invention may, of course, be carried out in other specific ways than those herein set forth without departing from the scope and the essential characteristics of the invention. The present embodiments are therefore to be construed in all aspects as illustrative and not restrictive and all changes coming within the meaning and equivalency range of the appended claims are intended to be embraced therein.

### Artificial Christmas Tree

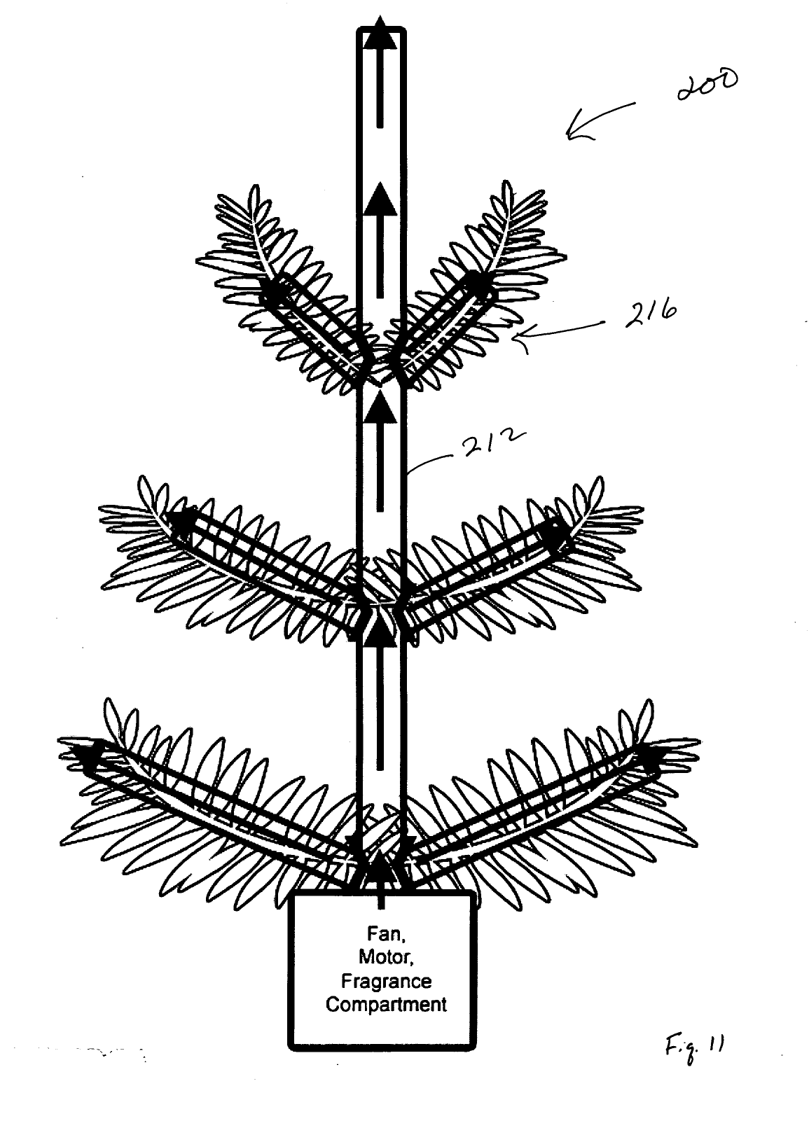

With further reference to the drawings, an artificial Christmas tree of the present invention is shown therein and indicated generally by the numeral 200. See FIGS. 11-13. Artificial Christmas tree 200 includes a generally vertical hollow trunk or stem indicated generally by the numeral 212 and a series of branches 216 secured to or extending from the trunk 212. In the context of this application, the term “artificial” simply means non-living. Thus, the artificial Christmas tree can be made of various materials such as plastic, metal, synthetic materials or could comprise dried Christmas trees or dried vegetation.

As shown in the drawings, the trunk 212 is hollow. Trunk 212 includes a surrounding wall structure 214 and includes a lower portion 214A and an upper portion 214B. As seen in the drawings, the lower portion 214A of the Christmas tree 212 is adapted to be supported by a base 220. That is, the Christmas tree 200 can be inserted into the base and supported in an upright manner. Base 220 may comprise an open top box or container and support materials such as Styrofoam, potting soil, dirt or other supporting structure. Alternatively, the lower portion 214A of the trunk may be formed into a bulb shape.

It is noted that the upper portion 214B of the trunk 212 extends above the uppermost branches 216. The upper end of the trunk may be open or partially closed. In the embodiments illustrated herein, the upper end of the trunk 212 includes a rounded end provided with a series of openings therein. As will be described subsequently herein, the trunk 212 is designed such that air and a fragrance can move upwardly through the same, and in at least on embodiment, is designed such that a fragrance can be emitted or dispersed from the upper portion 214B of the trunk into an environment where the artificial Christmas tree 200 resides. Also, it is appreciated that the wall structure 214 of the trunk 212 may include one or more openings 214C selectively placed along the length of the trunk. Again, as will be appreciated from subsequent portions of this disclosure, openings 214C within the trunk may permit air to enter the trunk 212 and move upwardly through the hollow trunk.

The Christmas tree branches 216 are disposed along the length of the trunk. Branches 216 form a part of the overall Christmas tree 200. It is appreciated that the branches 216 may be secured or integrally formed with the trunk 212 through various manufacturing and fabrication techniques. Branches 216 may also assume various shapes and configurations. In some embodiments, each branch would include a supporting structure that extends outwardly from the trunk 212 and an array of stem or sub branches having artificial Christmas tree like needles thereon.

The present invention also entails associating a fragrance source indicated generally by the numeral 222 with the Christmas tree 200 for dispersing a fragrance into the environment. The fragrance source 222 can be of various conventional types. Further, the particular scent emitted by the fragrance source 222 may vary and may be selected to simulate or mimic the smell or scent of various types of Christmas trees. The size of the fragrance source 222 and/or the size of the air intake openings and/or outlet openings formed in the Christmas tree 200 may affect the intensity of the fragrance dispersed into the environment.

Various structures, systems and methods can be employed for dispersing the fragrance from the artificial Christmas tree 200. These have been disclosed in the parent applications: U.S. patent application Ser. No. 10/164,818, filed Jun. 7, 2002 and entitled “Artificial Flower,” and U.S. patent application Ser. No. 10/756,224 filed Jan. 13, 2004 and entitled “Artificial Flower.” These disclosures are expressly incorporated herein by reference. In these applications, various artificial flowers have been disclosed and wherein the artificial flowers are provided with means for emitting a fragrance. Various types of fragrance delivery systems have been disclosed in connection with these artificial flowers. The same fragrance delivery systems are applicable to the artificial Christmas tree 200 of the present invention and therefore details of such will not be dealt with herein. However, as discussed in the two patent applications incorporated herein, the fragrance source may assume various forms including a liquid, a gas or a solid. Also, a fan can be incorporated into the system for moving air past the fragrance and through the artificial tree 200. Further, as discussed in the case of the Artificial Flower, a source of heat may be utilized in conjunction with the fragrance source to facilitate the emission of the fragrance from the fragrance source.

In addition, as disclosed with respect to the Artificial Flower, the fragrance source may be positioned at various locations with respect to the Christmas tree 200. The fragrance source may be contained in a container which may preferably be disposed adjacent the Christmas tree and provided with communication or conduit means for channeling the fragrance from the container to the Christmas tree. In addition, the fragrance source can be stationed in the Christmas tree itself, again at various locations in the trunk 212, for example.

In the embodiment illustrated herein, the fragrance-laden air generally passes upwardly through the trunk 212 of the Christmas tree. It is appreciated, however, that the branches may be provided with conduits or air passageways that would communicatively connect to the trunk 212. This would permit the fragrance—air mixture to be directed not only upwardly through the trunk 212, but through the branches 216 as well. The conduits or air passageways in the branches would be provided with outlets to enable the fragrance—air mixture to be emitted there from.

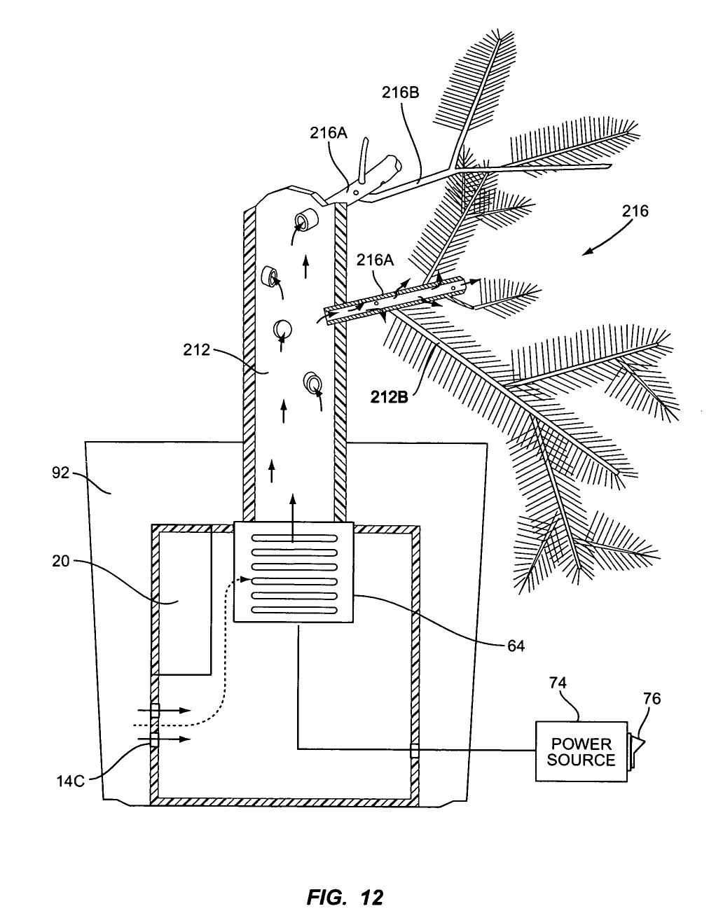

In this regard, reference is made to FIG. 12. FIG. 12 is a fragmentary view illustrating one example of how detachable branches 216 could be connected to the trunk 212. Further, FIG. 12 shows an example of how the air-fragrance mixture could be directed through and out the branches 216. It is to be appreciated that this is simply one example of how the branches 216 could be structured and connected to the trunk 212. There are other ways of accomplishing the same. In any event, in the embodiment illustrated in FIG. 12, the trunk includes a series of openings. There is provided a series of branches 216A that can be inserted into the openings within the trunk 212. These branches 216A may include sub-branches or extensions 216B. Further, note that the branch segments 216A include an elongated opening passing from end to end. This permits the air-fragrance mixture passing through the trunk to actually pass through the branch segments 216A. Further, the branch segments 216A include smaller side ports for allowing the air-fragrance mixture to be emitted therefrom.

As noted above, there are various ways to direct the air-fragrance mixture through the artificial Christmas tree 200. One particular way is similar to that shown in FIG. 6 with respect to the artificial flower. An example of that type of application is shown in FIG. 12. Note that the Christmas tree 200 is set within a pot or container 92. Disposed within the pot or the container is a compartment or receptacle 88 that includes the fragrance source 20 and a number of air inlet openings 14C. A fan 64 is associated with the box 88 and is powered by power source 74 having a switch 76. In this particular example, air is pulled in through inlets 14C and passed over the fragrance source 20. As air passes over the fragrance source 20, the fragrance mixes with the air to form an air-fragrance mixture. This mixture is channeled through openings within the fan housing 64. From there the fan directs the air-fragrance mixture up through the trunk 212 of the artificial Christmas tree 200.

From the foregoing specification and discussion, it is appreciated that the artificial Christmas tree of the present invention has the advantage of emitting a fragrance or scent that simulates or mimics a live Christmas tree.

The present invention may, of course, be carried out in other specific ways than those herein set forth without departing from the scope and the essential characteristics of the invention. The present embodiments are therefore to be construed in all aspects as illustrative and not restrictive and all changes coming within the meaning and equivalency range of the appended claims are intended to be embraced therein.

1. An artificial Christmas tree comprising:
a. a trunk having an air passageway formed therein;
b. a series of Christmas tree branches adapted to extend from the trunk; and
c. a fragrance source associated with the Christmas tree for emitting a fragrance.

2. The artificial Christmas tree of claim 1 further including diffuser to move air across the fragrance source.

3. The artificial Christmas tree of claim 2 wherein the diffuser comprises a fan.

4. The artificial Christmas tree of claim 2 wherein the diffuser comprises a heat source.

5. The artificial Christmas tree of claim 2 where at least a portion of the diffuser is disposed within the trunk of the artificial Christmas tree.

6. The artificial Christmas tree of claim 5 wherein the diffuser is disposed within an upper portion of the trunk.

7. The artificial Christmas tree of claim 1 wherein the fragrance source includes a container having a fragrance contained therein.

8. The artificial Christmas tree of claim 7 where the container includes one or more openings and the fragrance comprises a fragrance block.

9. The artificial Christmas tree of claim 1 wherein the fragrance source is disposed within the artificial Christmas tree.

10. A method of generating a fragrance for an artificial Christmas tree having a trunk and branches extending therefrom comprising: associating a fragrance source with the artificial Christmas tree and emitting a fragrance from the source such that the fragrance imparts a scent about the artificial Christmas tree.

11. The method of claim 10 comprising directing air across the fragrance source to emit a fragrance from the artificial Christmas tree.

12. The method of claim 11 wherein directing air across the fragrance source comprises locating a fan within a portion of the artificial Christmas tree and directing air from the fan across the fragrance source.

13. The method of claim 12 comprising locating the fan within the trunk of the artificial Christmas tree.

14. The method of clam 11 further comprising locating a heater within at least a portion of the trunk and directing air heated by the heating source across the fragrance source.

15. The method of clam 11 wherein associating a fragrance source with the artificial Christmas tree comprises locating a fragrance container within or adjacent the artificial Christmas tree, said fragrance container having a fragrance contained therein.

16. The method of claim 15 further comprising replacing the fragrance source by refilling the fragrance container with fragrance.
